(19) United States
(12) Patent Application Publication
(10) Pub. No.: US 2005/0026135 A1
(43) Pub. Date: Feb. 3, 2005

(54) Method for rapid detection of microorganisms by changing the shape of micro colonies

(76) Inventor: Sergey Gazenko, Mason, OH (US)

Correspondence Address: Sergey Gazenko, 1106 Park Side Ln., Mason, OH, 45040, US

(21) Appl. No.: 10/628,110

(22) Filed: Jul. 28, 2003

(51) Int. Cl. C12Q 1/04

(52) U.S. Cl. 435/4; 435/34

(57) ABSTRACT

The time of the detection and enumeration of microorganisms after their growth on solid or liquid nutrient media depends on the visibility of colony or suspension by naked eye or optical instruments. Visibility depends mainly on light absorbance by layer of cells in colony or suspension. The growth of microorganisms in micro channels needs much less amount of cells to reach the same light absorbance as done by regular growth. Smaller amount of cells needs shorter time for their reproduction. Therefore detection and enumeration of cells could be done in several times faster than by previously known growth methods. Also the time of detection and enumeration could be shortened by additional usage of chemical substances or physical factors that increase light absorbance or instill fluorescence. To reach needful light absorbance the volume of one micro channel must be extremely small—only in several thousands times larger than the volume of one cell and longevity of channel must be in several times longer than diameter of a channel.

### BACKGROUND OF THE INVENTION

The growth of microorganisms in order to detect, enumerate and identify livable cells—bacteria, fungi, actinomycetes—is one of the most widely used methods in microbiology. The growth occurs on either solid or liquid artificial or natural nutrient media. Hundreds of different media for total count and growing of groups or species of microorganisms are known currently. It usually takes from several hours to several days to form well visible colonies or suspension of cells. Improvement of visibility (detectability) of colonies could shorten the time between inoculation and detection of the colony.

There are several different methods, instruments and devices employed to enhance colony visibility. Thus the addition of special, non toxic for cells substances (some artificial chromogenic or fluorogenic substrates) to solid nutrient media could change color of the colonies or make them fluorescent and improve colonies visibility on early stages. Toxic artificial substrates (example: Tetrazolium salts) or other substances could be used on late stage of colony or suspension of cell growth to colorize cells and make them more clearly visible.

Detection and enumeration of the colonies are done visually or with magnifying devices. Visual detection and enumeration using magnifying glass requires relatively big colonies; from hundreds of microns to millimeters in diameter. Microscopy helps to find micro colonies with tens of microns in diameter. These colonies contain at least several hundreds of cells and need at least 5-10 hours of incubation to form colony of this size.

Thus, modern microbiology utilizes three approaches to shorten time of needed growth and improve visibility of colonies; first—employ optimal growth nutrient media, second—addition of chemical matter in nutrient media or on a colony, or into suspension of cells, to change colonies optical characteristics, and third—employ optical instruments or devices.

There are no methods utilizing the shape of the colony during it growth in order to enhance its optical density (light absorbance). Changing of colony shape from regular semi-sphere with large volume and large amount of cell to thin cylinder shape with small volume and small amount of cells could strongly reduce the time between inoculation and colony counting. Smaller amounts of cells need shorter time for their production. The usage of chemicals producing color or fluorescence and optical instruments and devices together with detection of cylindrical colonies could improve visibility and reduce the time of analysis.

Reduction of the time between inoculation and detection is very important for early decision in quality and process control in food and biotechnological industry, medical microbiology and epidemiology, air, water and surfaces control of indoor and outdoor environment, and scientific research.

### SUMMARY OF INVENTION

The invention is based on growing of micro colonies in thin and long micro channels, instead of regular growth on flat surface of solid nutrient media, or flat surface of filter placed on nutrient media, or growth in relatively big volume of liquid nutrient media.

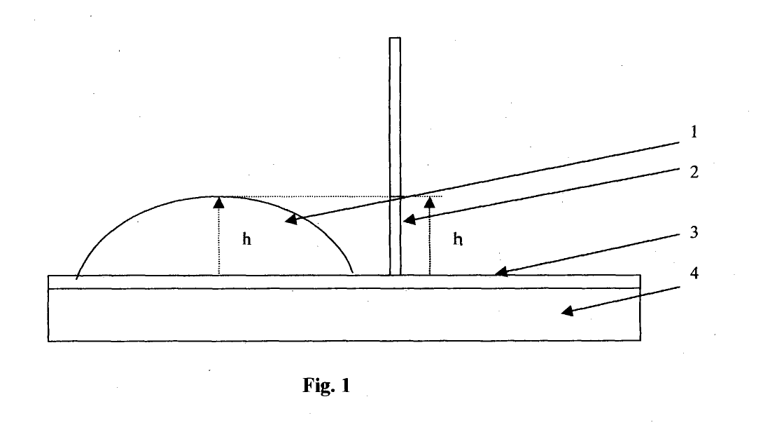

The shape of regular micro colony is usually semi-sphere. The thickness (height) of micro colonies is crucial to make it visible using microscope because thick (high) colony has larger light absorbance—most important optical characteristic of visibility. Long and thin micro colony have the same light absorbance as regular semi-sphere colony of the same height—h (see FIG. 1: 1—regular micro colony; 2—micro colony in micro channel; 3—filter; 4—nutrient media). The volume and amount of cells in long thin micro colony is much less, and therefore the time of incubation to create well visible colony needs to be much shorter.

The growth of a cylindrical micro colony could be done with a help of grid that has large amount of very small and long channels. The diameter of this channel needs to be very small, only in 4-20 times larger than the size of investigated cells. Good example for these purposes could be MCGP—Micro Channel Glass Plate. MCGP contain thousands of extremely small precisely itched long channels. Regular MCGP has a diameter of each channel 10 microns, length 500 microns, and the amount of channels is 700,000 per cm². Other grids or MCGP could be useful also.

Calculations below show obvious advantage in shortening of time of growth in micro channels in comparison with flat surface.

The regular shape of colonies growing on flat surface of solid nutrient media is, usually near to semi-sphere. The volume of semi-sphere is Vₛₛ=¶·h²·(R−h/3), where Vₛₛ—volume of semi-sphere, R—radius of sphere and h—part of radius—height of semi-sphere.

The volume of cylinder (cylindrical colony) is V_{cc}==¶·R²·h, where R—radius of cylinder, h—height of cylinder.

Micro colony with height (h) 10 μk and R=20 μk has volume:
Vₛₛ=3.14·10²·(20−10/3)=5234 μk³

Cylindrical colony with the same height (h=10 μk) and R=2.5 μk has volume:
V_{cc}=3.14·2.5²·10=196 μk³
Thus, the volume of a cylindrical colony is smaller than volume of semi-spherical micro colony with the same height in 27 times, and both have the same light absorbance.
The volume of one cell of Escherichia coli (E. coli) is near to 1 μk³. The speed of multiplying of E. coli is around 20 min at optimal temperature, on optimal media. One cell of E. coli can produce 8 cells in one hour, 64 in two hours, 512 in 3 hours, 4096 in 4 hours and 32768 in 5 hours. Thus, one micro colony contains 5234 cells could be formed in 4.2 hours. The cylindrical colony with the same height and light absorbance (196 cells) could be formed in 2.5 hours.
Therefore growth of micro colonies with cylindrical shape has significant advantage because of visualization of colony could be done at much earlier stages.

The visualization of microorganisms in micro channels filled by liquid nutrient media is much faster than in regular tubes or wells of immunological plate, or other known laboratory devices for microorganisms growth, because of very small volume of micro well and it long cylindrical shape. Thus, one cell in a cylindrical micro channel, with a length 500 μk and diameter 10 μk (V=40,000 μk³) correspond to concentration of 25 millions cells per ml (V=10¹² μk³). 40 cells in a micro channel correspond to the concentration 10\hat{ }9 cells per ml—well visible concentration. One cell of E. coli can reach this concentration (concentration 40 cells per micro channel=10\hat{ }9 cells per ml) in 1.7 hours.

Experiments show that 10 layers of colorless small cells (for example E. coli) are enough to find visual differences between micro channels contain cells and empty micro channels using regular light microscope with even small magnification of ×100. Smaller diameter of the channel needs smaller amount of cells to create 10 layers of cells in the channel.

Table 1 represent amount of layers of E. coli that could be produced in micro channels of different diameters in different time.

TABLE 1
Correlation between time of forming layers of cells in micro channels
and diameter of micro channel (E. coli, growth at 37° C. on TSA;
the time of multiplication = 20 min)

| Diameter of | Hours of Incubation | | | | |
|---|---|---|---|---|---|
| Micro channel | 1 hour | 2 hours | 3 hours | 4 hours | 5 hours |
| 2 μk | 3 layers | 21 | 171 | 1365 | 10920 |
| 3 μk | 1 | 9 | 73 | 585 | 4680 |
| 4 μk | 0.6 | 5 | 39 | 315 | 2520 |
| 5 μk | 0.4 | 3 | 26 | 205 | 1640 |
| 7 μk | 0.2 | 2 | 13 | 108 | 860 |
| 10 μk | 0.1 | 1 | 6 | 50 | 410 |

Table 1 shows that 10 layers of cells will be reached in micro channel with diameter 2 μk in 1.5 hours; in 3 μk micro channel in 2 hours; in 4 μk micro channel in 2.3 hours; in 5 μk micro channel in 2.7 hours; in 7 μk micro channel in 2.9 hours and in 10 μk micro channel in 3.5 hours. Thus, the detection and enumeration of long cylindrical micro colonies according this invention could be done in 10-20 times faster than regular growth, detection and enumeration of CFU.

The channels containing micro colony look like dark dots. Addition of artificial chromo- or fluorogenic substrates to micro colonies could reduce time between inoculation and detection: color or fluorescence could make micro colonies much more visible on earlier stage.

This invention differs from other methods of detection of CFU, by using of plate containing hundreds of thousands of extremely small and long channels (micro channel plate). The combination of micro channel plate and filter allows trapping of cell on the filter surface and growing colonies inside channel. Those colonies will obtain high cylindrical shape. High cylindrical shape of colony has long optical way (high light absorbance) but smaller volume and amount of cells which drastically reduces time of analysis. This method could be realized with a simple device consisting of a plate with channels, filter to trap cells by filtration from air or liquid and frame consists from several parts.

### DETAILED DESCRIPTION OF THE INVENTION

This invention is based on the method and device for trapping cells from liquids or air, grow relatively short time on solid nutrient media or in liquid nutrient media and find dark (not colored), colored or fluorescent channels that looks like large round dots under regular or fluorescent microscope. The time of analysis could be reduced, and sensitivity could be enhanced by the usage of channels of smaller diameter and substances produced color or fluorescence. Physical factors like heating in order to coagulate proteins and increase light absorbance or addition of the substances produced gas bubbles like O₂ produced from H₂O₂ by Catalase could be employed also.

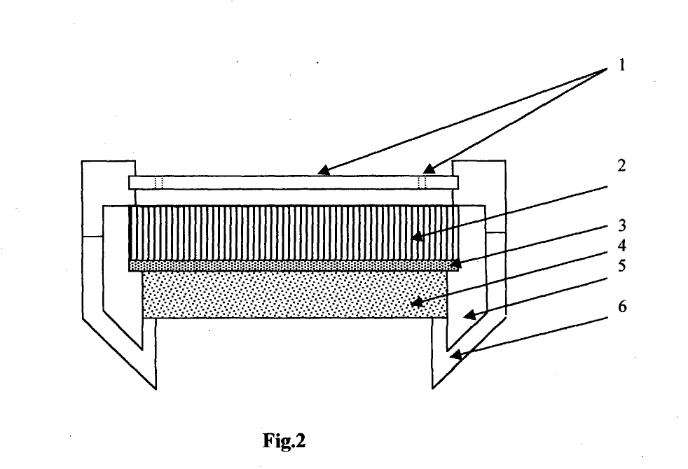

Simple device for trapping cells in the channels by filtration shown on FIG. 2. It consists from a lid (1) with transparent glass or plastic with one, two or more very small holes for respiration, micro channel plate (2), filter to restrain cells (3), holder for filter and micro channel plate, and porous support (4) for filter and micro channel plate adjusted to holder (6).

Procedure for sampling, growth and enumeration of the colonies is following:
Liquid or air sample containing microorganisms filtrated through the device. Cover lid (1) taken off before filtration, and special funnel for liquids (not shown on the picture) could be adjusted. During this process, cells if any are caught in some of the channels of micro channel plate (2) on the surface of the filter (3).
Support (4) adjusted to holder (6) removed.
Lid (1) placed to prevent further contamination.
Holder (5) with the micro channel plate (2) and the filter (3) placed on the surface of eligible solid nutrient media (not shown on the picture) or in the container with liquid nutrient media. Nutrient media wet filter and support the growth of cylindrical micro colony or penetrate through filter in a channels, and supports the growth of suspended microorganisms.
Device with nutrient media is incubated needful time at appropriate temperature. In order to cut time of analysis by increasing of light absorbance or add fluorescence, the device could be placed in the, container with eligible solution of artificial substrate. Otherwise this matter could be added to solid nutrient media in advance.
Device placed under light or fluorescent microscope and the amount of dark, colored or fluorescent channels detected and enumerated. This amount that corresponds to the amount of cells trapped on the surface of the filter.

This method and device could be used with a broad range of different solid and liquid natural or artificial media. Micro channel plate without a filter could be used to find contamination on surfaces by spraying liquid nutrient media on surface and placing micro channel plate above, incubate needful time and read results under microscope.

This invention could be realized in many different optical or opto-electronic instruments and devices.

1. Method for rapid detection of live cells by detection of micro colonies produced by these cells which method comprises:
growing of micro colonies in a small and thin channels of the device consisting from micro channel plate, filter to trap cells and frame in order to form long cylindrical micro colonies, as a result of growth on solid nutrient media in order to increase their visualization with optical instruments by changing optical characteristics of light passing through the channels, where channels contain micro colonies will look different from empty channels optical characteristics.

2. The method of claim 1 wherein micro colonies formed in long and thin channels with a shape another than cylinder.

3. The method of claim 1 wherein device placed in liquid nutrient media and trapped cell produces suspension of cells in a channel.

4. The method of claim 1 wherein micro channel plate filled by liquid nutrient media and placed on examined surface, or a surface covered by nutrient media and micro channel plate put on after.

5. The method of claim 1 wherein optical characteristics changed as a result of adding of artificial substrate produced colored or fluorescent substance or other substance to colorize cells or use physical methods to change optical characteristics of channels containing cells like heating to coagulate proteins, add hydrogen peroxide to produce micro bubbles or grow cells in highly colored liquid nutrient media and observe the increasing of light transmittance in the channels with growing cells.
